SEQUENCE LISTING (7 sequences)

SEQ ID NO: 1 - protein, 750 aa, synthetic construct
MRLKWHAALSATLVVSLMIGQLPASAKRETGRKGSLAIEAKEGLSLASQTSQKRSTELVVVQFDGPVEEEWKEQVEDLGVTLGDYIPDYAFIARVDGEKAQRALKKLSFVRNVLPFHPVSKVSPTLRSTLGKRADVDVAVIGFDRGVDMGRTLRRIVEEGDVEQVKSLEEAPHISMARLSGRSLEEVIQSDEVIAVVPVPERKQRNDRAAAIIRADDLASIGYTGEGQIVGVADSGLDTGDLDRIHPDFEGRVKELYAIGRKGDASDPDGHGTHVAGSIVGTGKASDGQYKGMAPDAKLVFHSMLDEYGYLAGDVSDILEEAYADGARIHSDSWGVDDDGEYGLDSFLFDQFLWEHPDMTALVAAGNAGVYGYETIGTPATAKNVIAVGASENDRPELDDDEADDPDEVAAFSSRGTTADGRLKPDLVAPGSYILSTRSSLAPAENFHGLFNASYAYLSGTSMATPVLAGGVAQVRQFLQENGVDNPSGALIKAMLISGADDLNEDMRVQGFGRANLRRAIETGFVDEKKGLKTGDVATYRLNVTDDSQPLAITLAWTDYPASLAAERTLVNNLNLRVVTPSGETLNGNDFFHYPYDDEVDNLNNVEQVWIKHPEEGVYTVTVEGYNVPMGPQPYALAANGELVSDNWQTVVKTGRLIEKSRKNRDHTYWIQAKQAGELNVTAEWDGDADLDLYVYDGDGNLIGSTDDEENPETLTVEMAEPGPYRIVIQLEEGKLARYRLQMTYPSRSR

SEQ ID NO: 2 - DNA, 2247 bp, synthetic construct
  source 1..2247: mol_type = other DNA
atgcgcttgaaatggcatgccgctttgagtgccaccttggttgttagtttgatgattggtcaattgccagccagtgccaaacgcgaaaccggtcgcaaaggcagtttggccattgaagccaaagaaggcttgagtttggctagtcaaaccagtcaaaaacgcagtaccgaattggttgttgttcaatttgatggcccagttgaagaagaatggaaagaacaagttgaagatttgggcgttaccttgggcgattatattccagattatgcctttattgctcgcgttgatggcgaaaaagcccaacgcgctttgaaaaagttgagttttgttcgcaatgtcttgccgtttcatccggttagtaaagttagtccaaccttgcgcagtacgttgggcaaacgcgctgatgttgatgttgccgttattggctttgatcgtggcgttgatatgggtcgcaccttgcgtcgcattgtcgaagaaggcgacgttgaacaagttaaaagtttggaagaagccccacatattagtatggctcgcttgagtggccgtagcttggaagaggttattcaaagtgatgaagttattgccgttgttccagttccagaacgcaaacaacgcaatgatcgcgctgccgctattattcgcgcagatgatttggccagtattggctataccggtgaaggccaaattgttggcgttgccgatagtggcttggatacaggcgatttggatcgcattcatccagattttgaaggccgtgtcaaagaattgtatgccattggccgtaaaggtgatgccagtgatccagatggccatggcacccatgttgctggcagtattgttggcaccggcaaagccagtgatggccaatataaaggtatggctccagatgccaaattggtctttcatagtatgttggatgagtatggctatttggctggtgatgttagcgatattctggaagaggcctatgccgatggcgcacgcattcatagtgatagttggggtgttgatgatgatggtgaatatggtttggatagcttcttgttcgatcagttcttgtgggaacatccagatatgaccgccttggttgccgctggcaatgctggcgtttatggctatgaaaccattggcaccccagccaccgccaaaaatgttattgctgttggcgctagtgaaaatgatcgcccagaattggatgatgacgaagccgatgatccggatgaagttgccgcttttagtagtcgtggcaccaccgctgatggccgtttgaaaccagatttggttgccccaggcagctatattttgagtacccgcagtagtttggctccagccgaaaactttcatggcttgtttaatgccagttatgcctatttgagtggcaccagtatggcaaccccagttttggctggcggtgttgcccaagttcgccaatttttgcaagaaaatggtgtcgataatccaagcggtgccttgattaaagccatgttgattagtggtgccgatgacttgaatgaagatatgcgcgttcaaggctttggccgtgctaatttgcgtcgcgctattgaaaccggctttgttgatgaaaagaaaggcctgaaaaccggtgatgttgctacctatcgcttgaatgttaccgatgatagtcaaccgttggccattaccttggcctggaccgattatccagccagtttggctgccgaacgcaccttggtcaataatttgaatttgcgcgttgttaccccaagtggcgaaaccttgaatggcaatgatttcttccattatccgtacgatgatgaagtcgataacttgaacaatgtcgaacaggtctggattaaacatccggaagaaggtgtttataccgttaccgttgaaggctataatgttccaatgggtccacaaccatatgccttggctgccaatggcgaattggtcagtgataattggcaaaccgttgttaaaacgggtcgcttgattgaaaagagtcgcaaaaatcgcgatcacacctattggattcaagccaaacaagcaggcgaattgaatgtcaccgctgaatgggatggtgatgctgatttggatttgtatgtttatgatggcgacggcaatttgattggcagcaccgacgatgaagaaaatccagaaacattgaccgtcgaaatggccgaaccaggtccatatcgcattgttattcaactggaagaaggcaaattggctcgctatcgtttgcaaatgacctatccaagtcgcagt

SEQ ID NO: 3 - protein, 750 aa, synthetic construct
MRLKWHAALSATLVVSLMIGQLPASAKRETGRKGSLAIEAKEGLSLASQTSQKRSTELVVVQFDGPVEEEWKEQVEDLGVTLGDYIPDYAFIARVDGEKAQRALKKLSFVRNVLPFHPVSKVSPTLRSTLGKRADVDVAVIGFDRGVDMGRTLRRIVEEGDVEQVKSLEEAPHISMARLSGRSLEEVIQSDEVIAVVPVPERKQRNDRAAAIIRADDLASTGYTGEGQIVGVADSGLDTGDLDRIHPDFEGRVKELYAIGRKGDASDPDGHGTHVAGSIVGTGKASDGQYKGMAPDAKLVFHSMLDEYGYLAGDVSDILEEAYADGARIHSDSWGVDDDGEYGLDSFLFDQFLWEHPDMTALVAAGNAGIYGYETIGTPATAKNVIAVGASENDRPELDEDEADDPDEVAAFSSRGTTADGRLKPDLVAPGSYILSTRSSLAPDENFHGLFNAFYAYLSGTSMATPVLAGGVAQVRQFLQENGVDNPSGALIKAMLISGADDLNEDMRVQGFGRANLRRAIETGFVDEKKGLKTGDVATYRLNVTDDSQPLAITLAWTDYPASLAAERTLVNNLNLRVVTPSGETLNGNDFFHYPYDDEVDNLNNVEQVWIKHPEEGVYTVTVEGYNVPMGPQPYALAANGELVSNNWQSVVKTGRLIEKSRKNRDHTYWIQAKQAGELNVTAEWDGDADLDLYVYDGDGNLIGSTDDEENPETLTVEMAEPGPYRIVIQLEEGKLARYRLQMTYPSRSR

SEQ ID NO: 4 - DNA, 2250 bp, synthetic construct
  source 1..2250: mol_type = other DNA
atgcgtcttaaatggcatgctgctttatcagctacacttgttgtctcacttatgattggtcaattaccagcttcagccaaacgtgaaacaggtcgtaaaggttcattagccattgaagctaaagaaggtctttcacttgctagccaaacaagccaaaaacgttcaacagaacttgtcgttgttcaatttgatgggccagttgaagaagaatggaaagaacaagttgaagatcttggtgttacacttggggattatattccagactatgcttttattgctcgtgtcgatggtgaaaaagctcaacgtgctcttaaaaaacttagctttgttcgtaacgttcttccatttcacccagtctcaaaagtttcaccaacacttcgttcaacacttggtaaacgtgctgatgttgatgttgccgttattggttttgatcgtggtgttgatatgggtcgtacattacgtcgtattgtagaagaaggtgacgtcgaacaagtcaaatcattagaagaagctccacacatttctatggctcgtttatcaggtcgttcattggaagaagttatccaatcagacgaagttattgccgttgttccagttccagaacgtaaacaacgtaatgatcgtgctgctgctattattcgtgcagatgatttagctagcacaggttatacaggtgaaggtcaaattgttggtgttgctgattcaggtttagacacaggtgatcttgatcgtattcatccagattttgaagggcgtgtcaaagaactttatgccattggacgtaaaggtgatgcttcagatccagatggtcatggtacacatgttgctggttcaattgttggaacaggtaaagcttcagacgggcaatataaaggtatggctccagatgctaaattggttttccattcaatgcttgacgaatacggttatcttgctggtgacgtttcagacattcttgaagaagcttacgctgacggggcacgtattcattctgattcatggggtgttgatgatgacggtgaatatggtcttgactcatttcttttcgaccaattcttatgggaacacccagatatgacagctttagttgctgctggtaatgctggtatttatgggtatgaaacaattggtacaccagccacagccaaaaatgttattgctgttggtgcttcagaaaacgatcgtccagaattagatgaagatgaagctgatgatcctgatgaagttgctgcattttcatcacgtggtacaacagctgatggtcgtttaaaaccagatcttgttgctccagggtcatacattttatcaacacgttcatcacttgccccagacgaaaatttccatggtctttttaatgccttctatgcctatctttcaggtacatctatggctacaccagttttagccggtggtgttgcacaagttcgtcaatttttacaagaaaatggggtcgacaatccatcaggtgctcttattaaagccatgcttatttcaggtgctgacgaccttaatgaagatatgcgtgttcaaggttttggtcgtgctaatttacgtcgtgctattgaaacaggatttgttgatgaaaaaaaaggtcttaaaacaggggacgttgccacctatcgtttaaatgttacagatgactcacaaccacttgccattacattagcttggacagattatccagcatcattagctgctgaacgtacccttgttaataaccttaatcttcgtgttgttaccccaagcggtgaaacattaaatgggaatgatttctttcattatccatatgatgatgaagtcgataatcttaataatgttgaacaagtctggattaaacatccagaagaaggggtttataccgttacagttgaagggtataatgttccaatgggtccacaaccatatgctttagctgctaatggtgaacttgttagcaataactggcaatcagttgttaaaaccggtcgtcttatcgaaaaatcacgtaaaaatcgtgaccacacctattggattcaagctaaacaagccggtgaacttaatgttactgctgaatgggatggtgatgcagatcttgacctttatgtttatgatggtgacgggaatcttattggatcaacagacgatgaagaaaatccagaaacacttactgttgaaatggctgaaccaggtccatatcgtattgtcattcaacttgaagaaggtaaacttgcccgttaccgtttgcaaatgacatatccatcacgttcacgt

SEQ ID NO: 5 - DNA, 2425 bp, synthetic construct
  source 1..2425: mol_type = other DNA
agcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccata

SEQ ID NO: 6 - DNA, 1296 bp, synthetic construct
  source 1..1296: mol_type = other DNA
aaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgactaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccagtttgttgaagattagatgctataattgttattaaaaggattgaaggatgcttaggaag

SEQ ID NO: 7 - DNA, 1460 bp, synthetic construct
  source 1..1460: mol_type = other DNA
aacggtgctctccaaatattcttatttagaaaagcaaatctaaaattatctgaaaagggaatgagaatagtgaatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggttacgtatttattaacttctcctagtattagtaattgtcggcactgaattcgagctcagcattattgagtggatgattatattccttttgataggtggtatgttttcgcttgaacttttaaatacagccattgaacatacggttgatttaataactgacaaacatcaccctcttgctaaagcggccaaggacgctgccgccggggctgtttgcgtttttgccgtgatttcgtgtatcattggtttacttatttttttgccaaagctgtaatggctgaaaattcttacatttattttacatttttagaaatgggcgtgaaaaaaagcgcgcgattatgtaaaatataaagtgatagcggtaccaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcttttgccgctagctaaaaaataaccaaaaagcaaggactgctgaaagggctgacataagccttttgccggcggtccttttttaattctgatttttca

(19) United States
(12) Patent Application Publication
(10) Pub. No.: US 2026/0327593 A1
(43) Pub. Date: Oct. 22, 2026

(54) ENZYME FOR HYDROLYZING COLLAGEN AND USE THEREOF

(71) Applicant: Hubei Kaitai Biotechnology Co., Ltd., Shiyan (CN)

(72) Inventors: KAI BAO, Shiyan (CN); RUOKUN WU, Shiyan (CN); CHUNQIANG HE, Shiyan (CN); ZIQI WU, Shiyan (CN); LILI BAO, Shiyan (CN); ZEHUI WU, Shiyan (CN); XIN FANG, Shiyan (CN)

(73) Assignee: Hubei Kaitai Biotechnology Co., Ltd., Shiyan (CN)

(21) Appl. No.: 19/531,669

(22) Filed: Feb. 6, 2026

(30) Foreign Application Priority Data
Dec. 9, 2024 (CN) 2024118023035

(51) Int. Cl. C12N 9/50; C12N 1/20; C12N 15/75; C12R 1/10; C12R 1/125

(52) U.S. Cl. CPC C12N 9/50; C12N 1/20; C12N 15/75; C12R 2001/10; C12R 2001/125

(57) ABSTRACT

The present application relates to the technical field of collagen, in particular to an enzyme for hydrolyzing collagen and use thereof. The enzyme has an amino acid sequence with at least 90% sequence identity to SEQ ID NO: 3. The enzyme is capable of hydrolyzing the collagen at an enzymatic hydrolysis condition of 80° C., has high enzyme activity, and also has hydrolytic activity on insoluble collagen.

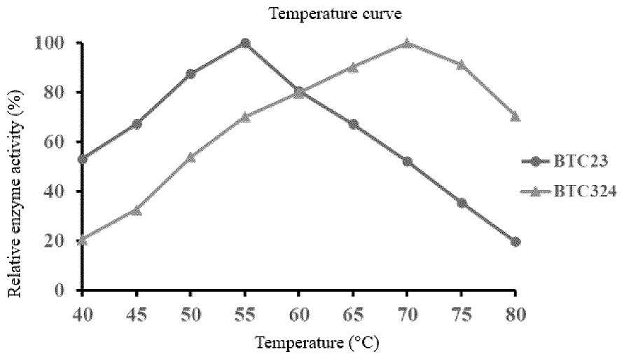

### CROSS-REFERENCE TO RELATED APPLICATIONS

The application claims priority to Chinese patent application No. 2024118023035, filed on Dec. 9, 2024, the entire contents of which are incorporated herein by reference.

### SEQUENCE LISTING

The sequence listing xml file submitted herewith, named “Sequence_Listing.xml”, created on Jan. 29, 2026, and having a file size of 18,114 bytes, is incorporated by reference herein.

### TECHNICAL FIELD

The present application relates to the technical field of collagen, in particular to an enzyme for hydrolyzing collagen and use thereof.

### BACKGROUND

Collagen is the most abundant structural protein in animals; and the collagen can be hydrolyzed to obtain collagen peptides with low molecular weights by means of acid-base hydrolysis, enzymatic hydrolysis, microbial fermentation, and other means. Compared to macromolecular collagen, the collagen peptides are more easily absorbed by the intestinal tract and have various physiological activities on the organisms, such as the anti-oxidant property, anti-tumor property, antibacterial property, immune regulation, and moisturizing. An enzymatic hydrolysis preparation method of the collagen peptides refers to specific hydrolysis of collagen by a single protease or a combination of multiple proteases, with a controlled process and a high yield of peptides, and is currently the most common way of large-scale production of collagen peptides. Collagen has a unique stable cross-linked triple helical structure and is mainly hydrolyzed by collagenases derived from mammals or bacteria, such as matrix metalloproteinases, serine proteinases, and metalloproteinases.

### SUMMARY

Thermally denatured collagen is more susceptible to hydrolysis under high-temperature enzymatic hydrolysis conditions, so that the high-temperature enzymatic hydrolysis conditions can improve hydrolysis efficiency of collagen and reduce the risk of microbial contamination during the enzymatic hydrolysis process. The embodiments of the present application provide an enzyme for hydrolyzing collagen, which is capable of hydrolyzing the collagen at an enzymatic hydrolysis condition of 80° C., has a higher enzyme activity, and also has hydrolytic activity on insoluble collagen.

To this end, the embodiments of the present application disclose at least the following technical solutions:

In a first aspect, the embodiments disclose an enzyme having an amino acid sequence with at least 90% sequence identity to SEQ ID NO: 3.

In a second aspect, the embodiments disclose a nucleic acid molecule having a nucleotide sequence as set forth in SEQ ID NO: 4.

In a third aspect, the embodiments disclose an expression vector including a nucleotide sequence as set forth in SEQ ID NO: 4.

In a fourth aspect, the embodiments disclose an expression strain which is a chassis cell carrying the expression vector according to the third aspect.

In a fifth aspect, the embodiments disclose a method of preparing the enzyme according to the first aspect. The method includes the following steps: obtaining the expression strain according to the fourth aspect; subjecting the expression strain to fermentation; and harvesting the enzyme from a fermentation broth.

In a sixth aspect, the embodiments disclose use of the enzyme according to the first aspect, the nucleic acid according to the second aspect, the expression vector according to the third aspect, and the expression strain according to the fourth aspect in preparing collagen peptides.

### BRIEF DESCRIPTION OF THE DRAWINGS

The FIGURE shows relative enzyme activity curves of enzymes BTC23 and BTC324 for enzymatic hydrolysis of casein at different enzymatic hydrolysis temperatures according to the embodiments.

### DETAILED DESCRIPTION OF THE EMBODIMENTS

To make objectives, technical solutions, and advantages of the present application more clearly, the following further describes the present application in detail with reference to the embodiments. It should be understood that the specific embodiments described herein are only intended to explain rather than limit the present application. Reagents not individually described in detail in the present application are all conventional reagents and are commercially available; and methods not specifically described in detail are routine experimental methods and can be known from the prior art.

The term “identity” (%) is used herein to refer to comparisons between polynucleotides or polypeptides and is determined by comparing two optimally aligned sequences in a comparison window, where portions of the polynucleotide or polypeptide sequences in the comparison window may include additions or deletions (i.e., gaps) as compared to the reference sequence for optimal alignment of the two sequences. The percentage may be calculated as follows: determining the number of positions at which the identical nucleic acid bases or amino acid residues occur in both sequences to yield the number of matched positions, dividing the number of matched positions by the total number of positions in the comparison window, and multiplying results by 100 to yield the percentage of sequence identity. Optionally, the percentage may be calculated as follows: determining the number of positions at which either the identical nucleic acid bases or amino acid residues occur in both sequences or the nucleic acid bases or amino acid residues are aligned with gaps to yield the number of matched positions, dividing the number of matched positions by the total number of positions in the comparison window, and multiplying results by 100 to yield the percentage of sequence identity. Those skilled in the art appreciate that there are many established algorithms that can be used for aligning two sequences. Optimal alignment of sequences for comparison may be conducted by any suitable method, including but being not limited to the local homology algorithm of Smith and Waterman (Smith and Waterman, Adv. Appl. Math., 2:482 [1981]), by the homology alignment algorithm of Needleman and Wunsch (Needleman and Wunsch, J. Mol. Biol., 48:443 [1970]), by the similarity search method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin software package), or by visual inspection, as is known in the art. Examples of algorithms suitable for determining the sequence identity percentage and the sequence similarity percentage include, but are not limited to, BLAST and BLAST 2.0 algorithm, and are described by Altschul et al. (Refer to Altschul et al., J. Mol. Biol., 215:403-410 [1990]; and Altschul et al., Nucl. Acids Res., 3389-3402 [1977], respectively). Software for performing BLAST analysis is publicly available through the website of the National Center for Biotechnology Information. This algorithm includes: first identifying a high scoring sequence pair (HSP) by identifying short words of length W in the query sequence, and the short words either match or satisfy some positive-valued threshold score T when aligned with a word of the same length in a database sequence. T is referred to as a neighborhood word score threshold (Refer to Altschul et al., supra). These initial neighborhood word hits act as seeds for initiating searches to find longer HSPs containing them. The word hits are then extended along two directions of each sequence to the extent that the cumulative alignment score cannot be increased. For nucleotide sequences, cumulative scores are calculated using parameters M (reward score for matching residue pairs; always >0) and N (penalty score for mismatching residues; always <0). For amino acid sequences, a scoring matrix is used for calculating the cumulative scores. Extension of word hits in each direction is halted when: the cumulative alignment score falls off by the quantity X from its maximum achieved value; the cumulative score reaches 0 or less than 0 due to the accumulation of one or more negative-score residue alignments; or the end of either sequence is reached. The BLAST algorithm parameters W, T, and X determine the sensitivity and speed of the alignments. The BLASTN program (for nucleotide sequences) uses the following as defaults: a word length (W) of 11, an expectation (E) of 10, M=5, N=−4, and a comparison of both strands. For amino acid sequences, the BLASTP program uses the following as defaults: a word length (W) of 3, an expectation (E) of 10, and the BLOSUM62 scoring matrix (Refer to Henikoff and Henikoff, Proc. Natl. Acad. Sci. USA 89:10915 [1989]). Exemplary determinations of the sequence alignment and the sequence identity percentage can use the BESTFIT or GAP programs in the GCG Wisconsin software package (Accelrys, Madison WI) and use default parameters provided.

The embodiments provide an enzyme capable of hydrolyzing collagen, which has good degrading activity on bovine bone type I insoluble collagen under high-temperature hydrolysis conditions without needing a swelling process. In addition, the collagen can be hydrolyzed at an enzymatic hydrolysis condition of 80° C., and it has high enzyme activity and also has hydrolytic activity on insoluble collagen.

In some embodiments, the enzyme has an amino acid sequence as set forth in SEQ ID NO: 3.

In one aspect, the embodiments disclose an enzyme having an amino acid sequence with at least 90% sequence identity to SEQ ID NO: 3.

In some embodiments, the enzyme has an amino acid sequence with at least 91%, 92%, 93%, 94%, 95%, 96%, 97%, 98%, or at least 99% identity to SEQ ID NO: 3.

In some embodiments, the enzyme has at least 99.1%, 99.2%, 99.3%, 99.4%, 99.5%, 99.6%, 99.7%, 99.8% or at least 99.9% identity compared to SEQ ID NO: 3.

In another aspect, the embodiments disclose a nucleic acid molecule having a nucleotide sequence as set forth in SEQ ID NO: 4. The nucleic acid molecule can be isolated free DNA, RNA, or a chimera of both, and is capable of being transcribed, translated, and expressed by an organism (e.g., a microorganism) to synthesize molecules of protein molecules (e.g., an enzyme as described above).

In another aspect, the embodiments disclose an expression vector including a nucleotide sequence as set forth in SEQ ID NO: 4.

In some embodiments, the expression vector further includes a base plasmid, and the base plasmid is pBT101.

In another aspect, the embodiments disclose an expression strain that is a chassis cell carrying the expression vector according to the third aspect.

In some embodiments, the chassis cell is selected from Bacillus subtilis, Bacillus lichenformis, or Bacillus amyloliquefaciens. In some embodiments, the Bacillus subtilis is Bacillus subtilis SCK6.

In another aspect, the embodiments disclose a method of preparing an enzyme provided by the above embodiments. The method includes: obtaining the expression strain provided by the above embodiments; subjecting the expression strain to fermentation; and harvesting the enzyme from a fermentation broth.

In another aspect, the embodiments disclose use of the enzyme according to the above embodiments, the nucleic acid according to the above embodiments, the expression vector according to the above embodiments, or the expression strain according to the above embodiments in preparing collagen peptides.

The present application will now be described in combination with more specific embodiments below, which are not to be construed as limiting the embodiments of the present application.

### Target Sequence and Expression Vector

The embodiments provide a preparing process of a base plasmid pTB101, including: performing PCR amplification on a sequence fragment including Escherichia coli replicon and an ampicillin resistance gene, as set forth in SEQ ID NO: 5; performing PCR amplification on a sequence fragment including a Bacillus replicon, as set forth in SEQ ID NO: 6; performing chemical synthesis on a sequence fragment including a tetracycline resistance gene, a promoter, a signal peptide, and a terminator, as set forth in SEQ ID NO: 7; performing seamless cloning linkage on the above three target DNA fragments; transforming a linkage product to Escherichia coli DH55a; screening positive colonies; and performing extraction from cultures of the positive colonies and sequence determination to obtain the base plasmid pTB101.

The embodiments provide DNA as set forth in SEQ ID NO: 4 as a target sequence. The target sequence may be obtained by PCR amplification and chemical synthesis, without limitations in the embodiments.

The embodiments also provide an expression vector pBT103 carrying the nucleotide sequence set forth in SEQ ID NO: 4. A preparing process of an expression vector includes: linking the DNA as set forth in SEQ ID NO: 4 with a base plasmid fragment (pBT101); transforming a linkage product to Escherichia coli DH55a; screening positive colonies; and performing extraction from cultures of the positive colonies and sequence determination to obtain the expression vector pBT103.

As a comparison, the comparative example also provides an expression vector pBT102 carrying the nucleotide sequence set forth in SEQ ID NO: 2. A preparing process of an expression vector includes: linking the DNA as set forth in SEQ ID NO: 2 with a base plasmid fragment (pBT101); transforming a linkage product to Escherichia coli DH55a; screening positive colonies; and performing extraction from cultures of the positive colonies and sequence determination to obtain the expression vector pBT102.

### Preparation of Expression Strain

The embodiments also provide an expression strain carrying the expression vector described above. A preparing method of the expression strain includes: transferring the expression vector provided in the above embodiments into Bacillus subtilis SCK6 (BNCC359202, Henan Beina Biological Inspection and Testing Co., Ltd.); screening positive colonies from transformants to obtain the expression strain.

The expression vector is transformed into the Bacillus subtilis SCK6, including:

Activate the strain Bacillus subtilis SCK6 with an LB solid plate, and cultivate at 37° C. for 12 h. Pick 2-3 single colonies into 10 mL of fresh LB fluid medium, and cultivate at 37° C., 220 r/min for 5-6 h, with a bacterial density OD₆₀₀ of about 0.8-1.0. Add a xylose solution with a final concentration of 1.5%, and continue an induction cultivation for 2 h to obtain competent cells of strain SCK6. Take 200 μL of competent cells to be evenly mixed with 10 μL of plasmid pBT103, perform a resuscitation cultivation for 1 h, coat a skimmed milk plate with a final tetracycline concentration of 10 g/mL, and cultivate at 37° C. for 12 h.

Positive colonies are screened from the transformants, including:

Pick transformants with the largest proteolytic circles on a transformation plate, and streak on the skimmed milk plate containing 10 g/mL tetracycline for purification to give single colonies with good hydrolytic activity, namely the expression strain B. subtilis KH103 for protease BTC324. As a control, the expression plasmid provided in the comparative example is transformed into Bacillus subtilis SCK6 to obtain the expression strain B. subtilis KH102 for the enzyme BTC23.

### Preparation of Enzyme for Hydrolyzing Collagen

The embodiments also provide a method of preparing an enzyme for hydrolyzing collagen. The method includes: obtaining the expression strain provided by the above embodiments; subjecting the expression strain to fermentation; and harvesting the enzyme from a fermentation broth.

In some embodiments, 3-5 single colonies of expression strains KH102 and KH103 are inoculated in 20 mL of LB solution containing 10 μg/mL tetracycline, and cultivated at 37° C., 220 r/min for 6-7 h, with a bacterial density OD₆₀₀ of about 1.0-1.2. 6 mL of seed solution is transferred to 300 mL of fermentation medium (1% of yeast powder, 2% of tryptone, 0.2% of sodium chloride, 2% of glucose, 1% of dipotassium hydrogen phosphate), cultivated at 37° C., 220 r/min for 60 h, the fermentation broth is collected for 8000 g centrifugation for 10 min, and a supernatant enzyme is collected to obtain the enzyme solution containing the target protease. The enzyme prepared by the expression strain KH103 provided in the embodiments is BTC324, and the enzyme prepared by the expression strain KH102 provided in the embodiments is BTC23.

### Enzymatic Hydrolysis Temperature

The casein hydrolytic activity of proteases BTC23 and BTC324 at 40° C., 45° C., 50° C., 55° C., 60° C., 65° C., 70° C., 75° C., 80° C., respectively, is determined with reference to “GB/T 23527-2009 Protease Preparation” at pH 9.0. As shown in FIGURE, the optimal enzymatic hydrolysis temperature of the enzyme BTC23 is 55° C., and the optimal enzymatic hydrolysis temperature of the enzyme BTC324 is increased to 70° C., thereby indicating that the enzyme BTC324 is more heat resistant and more favorable for enzymatic hydrolysis of bovine bone insoluble collagen under high-temperature conditions.

### Enzymatic Hydrolysis of Bovine Bone Type I Insoluble Collagen

### 1. Collagen Activity

The activity of BTC23 and BTC324 on bovine bone type I insoluble collagen is determined by a ninhydrin chromogenic method (Nagano et al. 2000). An enzyme activity unit is defined as one enzyme activity unit U per mg of protein required to produce 1 μmol leucine per mg of insoluble collagen degraded per hour at a specific temperature.

Determination of enzyme activity at 55° C.: 800 μL of 50 mmol/L Tris-HCl (pH 9.0), 4 mmol/L CaCl₂, and 10 mg of bovine bone type I insoluble collagen (ABM-5162, Wuhan Amyjet Scientific Inc.) substrate, shaking and even mixing, and water bath at 55° C. for 30 min. 200 μL of BTC324 enzyme solution diluted to a protein amount of 1 mg with 50 mmol/L Tris-HCl (pH 9.0) is added, and water bathing, shaking and enzymatic hydrolysis are performed at 55° C. for 2 h, and a reaction is stopped by adding 1 mL of 0.1 mol/L acetic acid. 10000 g centrifugation is performed for 10 min to obtain an enzymatic hydrolysate aqueous solution. 40 μL of enzymatic hydrolysate aqueous solution and 200 μL of ninhydrin-sodium citrate mixed liquor are taken, and are evenly mixed and boiled for 20 min, 1 mL of 50% n-propanol solution is added after cooling to room temperature, even mixing is performed, 200 μL is taken to an elisa plate, and a light absorption value is determined at 600 nm with a microplate reader. The enzyme activity of wild-type BTC23 is determined synchronously. To eliminate background effects of the solution in the detection system, a blank control group is set. The blank control group is that 200 μL of boiled and inactivated diluted enzyme solution is reacted with 10 mg of bovine bone insoluble collagen, and the remaining is same as that of the experimental group. 40 μL of leucine solutions with different concentrations are reacted with the ninhydrin-sodium citrate mixed solution to determine the light absorption value at 600 nm to create a standard curve. The determination method of enzyme activity under conditions of 60° C., 65° C., 70° C., 75° C., and 80° C. refers to that at 55° C.

TABLE 1

| Temperature | Enzyme activity of | Enzyme activity of |
|---|---|---|
| (°C) | BTC23 (U/mg) | BTC324 (U/mg) |
| 55 | 678.3 | 702.2 |
| 60 | 642.7 | 787.3 |
| 65 | 492.5 | 955.7 |
| 70 | 343.4 | 1135.1 |
| 75 | 228.2 | 902.4 |
| 80 | 75.1 | 633.6 |

As shown in Table 1, the enzyme activity of both enzyme BTC23 and enzyme BTC324 showed a trend of increasing and then decreasing to different extends with an increasing temperature from 55° C. to 80° C., but the optimal enzymatic hydrolysis temperature of the enzyme BTC23 is 55° C., and the optimal enzymatic hydrolysis temperature of the enzyme BTC324 is 70° C. In addition, for the enzyme activity at the respective optimal enzymatic hydrolysis temperatures, the enzyme BTC324 is significantly higher than the enzyme BTC23, thus indicating that the enzyme BTC324 is more beneficial for high-temperature enzymatic hydrolysis of the bovine bone type I insoluble collagen.

### 2. Preparation of Enzymatic Hydrolysate of Bovine Bone Type I Insoluble Collagen

In the embodiments, the BTC23 and BTC324 enzymes are also used for hydrolyzing the bovine bone type I insoluble collagen to obtain a BTC23 enzymatic hydrolysate and a BTC324 enzymatic hydrolysate.

Specifically, 10 mg of bovine bone type I insoluble collagen is dissolved in 2 mL of 50 mmol/L Tris-HCl (pH 9.0) solution at 70° C., the enzyme BTC324 or BTC23 accounting for 0.8‰ (W/W) of the amount of collagen is added, and shaking is performed for reacting for 2 h, 3 h, and 4 h. Heating is performed to 90° C.-100° C., treatment is performed for 10 min to inactivate the enzyme, and 10000 g centrifugation is performed for 10 min to obtain the BTC23 enzymatic hydrolysate and the BTC324 enzymatic hydrolysate.

The ratio of the amino nitrogen content of the BTC23 enzymatic hydrolysate and the BTC324 enzymatic hydrolysate to the total nitrogen content of the substrate is determined, the degree of hydrolysis (DH) of the substrate protein is calculated (Lu Chen et al. 2024), and the enzymatic hydrolysis performance of the mutant BTC324 to the bovine bone insoluble collagen is evaluated. The total nitrogen content in bovine bone insoluble collagen is determined by the Kjeldahl method with reference to GB 5009.5-2016 “Determination of Protein in Food in National Food Safety Standards”. The content of nitrogen in the amino state in the enzymatic solution is determined by autopotentiometric titration method of formaldehyde, and the results are shown in Table 2 below.

TABLE 2

| Enzymatic | DH of BTC23 | DH of BTC324 |
|---|---|---|
| hydrolysis time (h) | (%) | (%) |
| 2 | 63.1 | 82.9 |
| 3 | 65.6 | 84.5 |
| 4 | 66.2 | 86.2 |

As shown in Table 2, after 2 h of enzymatic hydrolysis at an enzymatic hydrolysis temperature of 70° C., the degree of hydrolysis of the enzyme BTC324 to the substrate reaches 82.9%, which is 131.4% of that of the enzyme BTC23. After 3 h of enzymatic hydrolysis, the degree of hydrolysis of the enzyme BTC324 is increased to 128.9% of that of the enzyme BTC23.

It is thus indicated that the enzyme BTC324 has excellent heat resistance, is capable of performing enzymatic hydrolysis on bovine bone insoluble collagen under high enzymatic hydrolysis temperature conditions, and has significant economic value for downstream applications.

### 3. Preparation of Bovine Bone Collagen Peptides

The bovine bone collagen solution is prepared with a substrate concentration of 6%-8% (W/V), and the pH is adjusted to 9. At the temperature of 70° C., the BTC324 enzyme solution accounting for 0.8‰ (W/W) of the amount of collagen is added, stirring is performed for reacting for 2 h, and the pH of the reaction process is adjusted to keep at 9.0 using a NaOH solution. The enzymatic hydrolysis solution is cooled to 50° C., the pH is adjusted to 6.5, neutral protease (50,000 U/mg) accounting for 0.2‰ (W/W) of collagen is added, and stirring is continued for reacting for 1 h. The enzymatic hydrolysis solution is heated to 90° C.-100° C. and treated for 20 min, the enzyme is inactivated, and enzymatic hydrolysis is stopped to obtain a crude enzymatic hydrolysis solution of the bovine bone collagen peptide. Adsorption decolorization and debittering are performed by adding activated carbon at a ratio of 1%-1.5% (w/v) and stirring for 60 min, and the activated carbon is removed by ultrafiltration through a ceramic membrane to obtain a clear solution of the bovine bone collagen peptide. The fraction of peptide segments with different molecular weights in the enzymatic hydrolysis solution is determined with reference to GB 31645-2018, and the results are shown in Table 3 below.

TABLE 3

| | | Number- | |
|---|---|---|---|
| | Percentage of | average | Weight-average |
| Molecular | peptide | molecular | molecular |
| weight (Da) | segments % | weight | weight |
| >3000 | 4.2 | 3362 | 3421 |
| 3000-2000 | 6.1 | 2354 | 2471 |
| 2000-1000 | 7.5 | 1426 | 1603 |
| 1000-500 | 46.7 | 680 | 713 |
| <500 | 35.5 | 245 | 263 |

The above is only the preferred specific implementation method of the present application, but the scope of protection of the present application is not limited to this. Any changes or replacements that can be easily thought by those skilled in the art within the scope of disclosure of the present application should fall within the scope of protection of the present application.

What is claimed is:

1. An enzyme having an amino acid sequence as set forth in SEQ ID NO: 3.

2. A nucleic acid molecule having a nucleotide sequence as set forth in SEQ ID NO: 4.

3. An expression vector, comprising a nucleotide sequence as set forth in SEQ ID NO: 4.

4. An expression strain, being a chassis cell carrying the expression vector according to claim 3.

5. The expression strain according to claim 4, wherein the chassis cell is selected from Bacillus subtilis, Bacillus licheniformis, or Bacillus amyloliquefaciens.

6. A method of preparing the enzyme according to claim 1, comprising:
obtaining an expression vector, wherein the expression vector comprises a nucleotide sequence as set forth in SEQ ID NO: 4;
obtaining an expression strain, wherein the expression strain is chassis cells carrying the expression vector;
subjecting the expression strain to fermentation; and
harvesting the enzyme from a fermentation broth.

7. The method according to claim 6, wherein the chassis cells are selected from Bacillus subtilis, Bacillus licheniformis, or Bacillus amyloliquefaciens.
